(19) United States
(12) Patent Application Publication
(10) Pub. No.: US 2008/0108535 A1
(43) Pub. Date: May 8, 2008

(54) COMPOSITION CONTAINING HOLLOW HEMISPHERICAL PARTICLES

(75) Inventor: Odile Aubrun-Sonneville, Antony (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/865,846

(22) Filed: Oct. 2, 2007

Related U.S. Application Data
(60) Provisional application No. 60/850,629, filed on Oct. 11, 2006.

(30) Foreign Application Priority Data
Oct. 4, 2006 (FR) 06 54079

(51) Int. Cl. A61K 8/30; A61Q 5/02; A61Q 19/00

(52) U.S. Cl. 510/122; 510/119; 510/130

(57) ABSTRACT

Composition containing (1) at least one foaming surfactant and (2) concave and/or annular particles of silicone material, in particular in the form of portions of hollow spheres, and (3) at least 20% by weight of water. The composition exhibits good foaming properties and gives in particular a fine and soft foam.

### REFERENCE TO PRIOR APPLICATIONS

This application claims priority to U.S. provisional application 60/850,629 filed Oct. 11, 2006, and to French patent application 0654079 filed Oct. 4, 2006, both incorporated herein by reference.

### FIELD OF THE INVENTION

The present invention relates to a composition, preferably one for topical application, comprising foaming surfactants and concave or annular particles of silicone material, and the use of said composition for example in the cosmetics field, in particular for the cleaning of keratinous substances, such as the skin and the hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The cleaning of the skin is very important for the care of the face and has to be as effective as possible as fatty residues, such as excess sebum, the residues from cosmetic products used daily and make-up products, accumulate in the skin folds and can block the pores of the skin and result in the appearance of spots. One means for thoroughly cleaning the skin is to use foaming cleaning products. The aim in general is to have available products giving a large volume of foam, a sign of good cleaning effectiveness.

The use is known, as cleaning products for the skin, of foaming gels. These products are solutions of foaming surfactants used for their detergent properties, these solutions being thickened by polymers. These gels can also comprise additives which make it possible to improve the quality of the foam, in particular its staying power, in order to facilitate the handling thereof, and also its softness, synonymous with care of and respect for the skin for users who wish to use comfortable products which do not leave a feeling of tightness or dryness. However, these additives are often film-forming, which can give an impression of imperfect cleaning.

### SUMMARY OF THE INVENTION

A need thus remains for a cosmetic and/or dermatological cleaning composition which is in the form of a gel and which at the same time has a good foaming performance and is comfortable.

The inventor has discovered, surprisingly, that the use of hollow hemispherical particles of micrometric size in foaming cleaning gels improves the quality of the foam, in particular its staying power and its softness.

### BRIEF DESCRIPTION OF THE DRAWING

FIG. 1 shows a concave particle in the form of portions of spheres with the shape of a bowl, illustrated in transverse cross section.

### DETAILED DESCRIPTION OF THE PREFERRED EMBODIMENTS

A subject-matter of the present invention is an aqueous composition preferably suitable for topical application comprising (1) at least one foaming surfactant, the amount of foaming surfactant(s) being sufficient for the composition to have a foaming effect (i.e., an effective amount), (2) concave and/or annular particles of silicone material, in particular in the form of portions of hollow spheres, and (3) at least 20% by weight of water, with respect to the total weight of the composition.

As the invention composition is preferably intended for topical application, it preferably comprises a physiologically acceptable medium. The term “physiologically acceptable medium” is understood here to mean a medium compatible with the skin, mucous membranes, nails, scalp, hair and/or eyes. It is preferably a cosmetically and/or dermatologically acceptable medium, that is to say which, in addition, exhibits a pleasant colour, a pleasant odour and a pleasant feel and which does not result in unacceptable discomfort (smarting, tightness, red blotches) liable to dissuade the consumer from using this composition. The composition of the invention can constitute in particular a cosmetic or dermatological composition.

According to a preferred embodiment of the invention, the composition is devoid of oil. The term “oil” is understood to mean any lipophilic compound which is liquid at ambient temperature (20-25° C.) and the term “devoid of oil”, is understood to mean that the composition comprises less than 2% by weight of oil, including less than 1.5, 1 and 0.5% by weight oil, and preferably 0% of oil.

The composition according to the invention can be provided in any form, but the forms of a solution, milk, cream and gel are preferred. The viscosity of the composition, measured using a Rheomat 180 device at 200 rpm at 25° C., can range, for example, from 0.5 to 1000 Pa·s and preferably from 0.8 to 500 Pa·s. The Rheomat 180 is equipped with a different spindle depending on the viscosities, for example with a spindle 3 for the range of viscosities from 0.2 to 4 Pa·s and with a spindle 4 for the range of viscosities of greater than 2 Pa·s. The viscosity is preferably measured 10 minutes after setting the spindle in rotation.

The aqueous composition of the invention comprises an aqueous medium. The term “aqueous medium” denotes a medium comprising at least water and optionally one or more other ingredients such as water-soluble organic solvents. In this aqueous medium, the amount of water is at least 20% by weight, with respect to the total weight of the composition. The compositions according to the invention can preferably comprise at least 30% by weight, indeed even at least 40% by weight, of water and preferably at least 50% by weight of water, and more, with respect to the total weight of the composition. They can comprise, for example, from 20 to 90% by weight, preferably from 30 to 85% by weight, better still from 40 to 80% by weight, with respect to the total weight of the composition.

Advantageously, the pH of the aqueous medium is compatible with keratinous substances and in particular with the skin. This pH preferably ranges from 3 to 8.5, better still from 3.5 to 8, preferably from 4 to 8.

While not bound by theory, it is believed that the presence of the concave and/or annular particles of silicone material makes it possible to confer improved staying power and improved softness, and thus properties of a comfortable feel, on the foam obtained by the composition according to the invention.

Another subject-matter of the invention is a process for improving the staying power and the softness of the foam obtained, from a composition for topical application comprising at least one foaming surfactant.

Concave and/or Annular Particles of Silicone Material

The concave and/or annular particles present in the composition according to the invention are silicone particles, in particular particles formed of portions of hollow spheres composed of a silicone material.

The particles preferably have a mean diameter of less than or equal to 10 μm, in particular ranging from 0.1 μm to 8 μm, preferably from 0.2 to 7 μm, more preferably ranging from 0.5 to 6 μm and preferably again ranging from 0.5 to 4 μm.

The term “mean diameter” is understood to mean the greatest dimension of the particle.

Advantageously, these particles have a density of greater than 1.

The portions of hollow spheres used in the composition according to the invention include those having the shape of truncated hollow spheres exhibiting a single orifice communicating with their central cavity and having a transverse cross section with the shape of a horseshoe or arch.

The silicone material is a crosslinked polysiloxane with a three-dimensional structure; it preferably comprises, indeed may even be entirely composed of, units of formula (I) SiO₂ and of formula (II) R¹SiO_{1.5} in which R¹ denotes an organic group having a carbon atom directly connected to the silicon atom.

The organic group R¹ can be a reactive organic group; R¹ can more particularly be an epoxy group, a (meth)acryloyloxy group, an alkenyl group, a mercaptoalkyl, aminoalkyl or haloalkyl group, a glyceroxy group, a ureido group or a cyano group and preferably an epoxy group, a (meth)acryloyloxy group, an alkenyl group or a mercaptoalkyl or aminoalkyl group. These groups generally comprise from 2 to 6 carbon atoms, in particular from 2 to 4 carbon atoms.

The organic group R¹ can also be an unreactive organic group; R¹ can then more particularly be a C₁-C₄ alkyl group, in particular a methyl, ethyl, propyl or butyl group, or a phenyl group and preferably a methyl group.

Mention may be made, as epoxy group, of a 2-glycidoxyethyl group, a 3-glycidoxypropyl group or a 2-(3,4-epoxycyclohexyl)propyl group.

Mention may be made, as (meth)acryloyloxy group, of a 3-methacryloyloxypropyl group or a 3-acryloyloxypropyl group.

Mention may be made, as alkenyl group, of the vinyl, allyl or isopropenyl groups.

Mention may be made, as mercaptoalkyl group, of the mercaptopropyl or mercaptoethyl groups.

Mention may be made, as aminoalkyl group, of a 3-[(2-aminoethyl)amino]propyl group, a 3-aminopropyl group or an N,N-dimethylaminopropyl group.

Mention may be made, as haloalkyl group, of a 3-chloropropyl group or a trifluoropropyl group.

Mention may be made, as glyceroxy group, of a 3-glyceroxypropyl group or a 2-glyceroxyethyl group.

Mention may be made, as ureido group, of a 2-ureidoethyl group.

Mention may be made, as cyano group, of the cyanopropyl or cyanoethyl groups.

Preferably, in the unit of formula (II), R¹ denotes a methyl group.

Advantageously, the silicone material comprises the units (I) and (II) according to a unit (I)/unit (II) molar ratio ranging from 30/70 to 50/50, preferably ranging from 35/65 to 45/55.

The particles of silicone material can in particular be capable of being obtained according to a process comprising:

(a) the introduction into an aqueous medium, in the presence of at least one hydrolysis catalyst and optionally of at least one surfactant, of a compound (III) of formula SiX₄ and of a compound (IV) of formula RSiY₃, where X and Y denote, independently of one another, a C₁-C₄ alkoxy group, an alkoxyethoxy group including a C₁-C₄ alkoxy group, a C₂-C₄ acyloxy group, an N,N-dialkylamino group including C₁-C₄ alkyl groups, a hydroxyl group, a halogen atom or a hydrogen atom and R denotes an organic group comprising a carbon atom connected directly to the silicon atom; and

(b) the operation in which the mixture resulting from stage (a) is brought into contact with an aqueous solution including at least one polymerization catalyst and optionally at least one surfactant, at a temperature of between 30 and 85° C., for at least two hours.

Stage (a) can be characterized as a hydrolysis reaction and stage (b) can be characterized as a condensation reaction.

In stage (a), the molar ratio of the compound (III) to the compound (IV) usually ranges from 30/70 to 50/50, advantageously from 35/65 to 45/55, and is preferably 40/60. The ratio by weight of the water to the total of the compounds (III) and (IV) preferably ranges from 10/90 to 70/30. The order of introduction of the compounds (III) and (IV) generally depends on their rate of hydrolysis. The temperature of the hydrolysis reaction generally ranges from 0 to 40° C. and usually does not exceed 30° C. in order to prevent premature condensation of the compounds.

For the X and Y groups of the compounds (III) and (IV):

mention may be made, as C₁-C₄ alkoxy group, of the methoxy or ethoxy groups;

mention may be made, as alkoxyethoxy group including a C₁-C₄ alkoxy group, of the methoxyethoxy or butoxyethoxy groups;

mention may be made, as C₂-C₄ acyloxy group, of the acetoxy or propionyloxy groups;

mention may be made, as N,N-dialkylamino group including C₁-C₄ alkyl groups, of the dimethylamino or diethylamino groups;

mention may be made, as halogen atom, of the chlorine or bromine atoms.

Mention may be made, as compounds of formula (III), of tetramethoxysilane, tetraethoxysilane, tetrabutoxysilane, trimethoxyethoxysilane, tributoxyethoxysilane, tetraacetoxysilane, tetrapropioxysilane, tetra(dimethylamino)silane, tetra(diethylamino)silane, silanetetraol, chlorosilanetriol, dichlorodisilanol, tetrachlorosilane or chlorotrihydrosilane. Preferably, the compound of formula (III) is chosen from tetramethoxysilane, tetraethoxysilane, tetrabutoxysilane and their mixtures.

The compound of formula (III) results, after the polymerization reaction, in the formation of the units of formula (I).

The compound of formula (IV) results, after the polymerization reaction, in the formation of the units of formula (II).

The R group in the compound of formula (IV) has the meaning as described for the R¹ group for the compound of formula (II).

Mention may be made, as examples of compounds of formula (IV) comprising an unreactive organic group R, of methyltrimethoxysilane, ethyltriethoxysilane, propyltributoxysilane, butyltributoxysilane, phenyltrimethoxyethoxysilane, methyltributoxyethoxysilane, methyltriacetoxysilane, methyltripropioxysilane, methyltri(dimethylamino)silane, methyltri(diethylamino)silane, methylsilanetriol, methylchlorodisilanol, methyltrichlorosilane or methyltrihydrosilane.

Mention may be made, as examples of compounds of formula (IV) comprising a reactive organic group R, of:
silanes having an epoxy group, such as (3-glycidoxypropyl)trimethoxysilane, (3-glycidoxypropyl)triethoxysilane, [2-(3,4-epoxycyclohexyl)ethyl]trimethoxysilane, (3-glycidoxypropyl)methyldimethoxysilane, (2-glycidoxyethyl)methyldimethoxysilane, (3-glycidoxypropyl)dimethylmethoxysilane or (2-glycidoxyethyl)dimethylmethoxysilane;
silanes having a (meth)acryloyloxy group, such as (3-methacryloyloxypropyl)trimethoxysilane or (3-acryloyloxypropyl)trimethoxysilane;
silanes having an alkenyl group, such as vinyltrimethoxysilane, allyltrimethoxysilane or isopropenyltrimethoxysilane;
silanes having a mercapto group, such as mercaptopropyltrimethoxysilane or mercaptoethyltrimethoxysilane;
silanes having an aminoalkyl group, such as (3-aminopropyl)trimethoxysilane, (3-[(2-aminoethyl)-amino]propyl)trimethoxysilane, (N,N-dimethylaminopropyl)trimethoxysilane or (N,N-dimethylaminoethyl)trimethoxysilane;
silanes having a haloalkyl group, such as (3-chloropropyl)trimethoxysilane or trifluoropropyltrimethoxysilane;
silanes having a glyceroxy group, such as (3-glyceroxypropyl)trimethoxysilane or di-(3-glyceroxypropyl)dimethoxysilane;
silanes having a ureido group, such as (3-ureidopropyl)trimethoxysilane, (3-ureidopropyl)-methyldimethoxysilane or (3-ureidopropyl)dimethylmethoxysilane;
silanes having a cyano group, such as cyanopropyltrimethoxysilane, cyanopropylmethyldimethoxysilane or cyanopropyldimethylmethoxysilane.

Preferably, the compound of formula (IV) comprising a reactive organic group R is chosen from silanes having an epoxy group, silanes having a (meth)acryloyloxy group, silanes having an alkenyl group, silanes having a mercapto group or silanes having an aminoalkyl group.

Examples of compounds (III) and (IV) which are preferred for the implementation of this invention are respectively tetraethoxysilane and methyltrimethoxysilane.

Use may independently be made, as hydrolysis and polymerization catalysts, of basic catalysts, such as sodium hydroxide, potassium hydroxide, sodium carbonate, sodium hydrogencarbonate, ammonia or amines, such as trimethylamine, triethylamine or tetramethylammonium hydroxide, or acidic catalysts, such as organic acids, for example citric acid, acetic acid, methanesulphonic acid, p-toluenesulphonic acid, dodecylbenzenesulphonic acid or dodecylsulphonic acid, or inorganic acids, such as hydrochloric acid, sulphuric acid or phosphoric acid.

When it is present, the surfactant used is preferably a nonionic or anionic surfactant or a mixture of the two. Sodium dodecylbenzenesulphonate can be used as anionic surfactant. The end of the hydrolysis is marked by the disappearance of the products (III) and (IV), which are insoluble in water, and the production of a homogeneous liquid layer.

The condensation stage (b) can use the same catalyst as the hydrolysis stage or another catalyst chosen from those mentioned above.

On conclusion of this process, a suspension in water of fine organosilicone particles is obtained, which particles can optionally be separated subsequently from their medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

The shape of the portions of hollow spheres obtained according to the above process and their dimensions will depend in particular on the method used to bring the products into contact in stage (b).

A somewhat basic pH and introduction under cold conditions of the polymerization catalyst into the mixture resulting from stage (a) will result in portions of hollow spheres with the shape of round-bottomed “bowls”, whereas a somewhat acidic pH and dropwise introduction of the mixture resulting from stage (a) into the hot polymerization catalyst will result in portions of hollow spheres having a transverse cross section with the shape of a “horseshoe”.

According to a preferred embodiment of the invention, portions of hollow spheres with the shape of “bowls” are used. These can be obtained as described in Application JP-A-2003-128788.

Portions of hollow spheres with the shape of a horseshoe are described in Application JP-A-2000-191789.

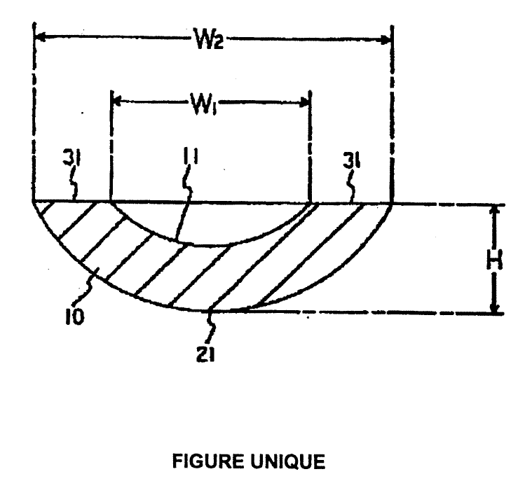

A concave particle in the form of portions of spheres with the shape of a bowl is illustrated in transverse cross section in the appended FIG. 1. The width W2 corresponds to the diameter of the particles.

As emerges from this FIGURE, these concave particles are formed (in cross section perpendicular to the plane of the opening delimited by the portion of hollow sphere) of a small internal arc (11), of a large external arc (21) and of segments (31) which connect the ends of the respective arcs, the width (W1) between the two ends of the small internal arc (11) ranging from 0.01 to 8 μm, preferably from 0.02 to 6 μm, on average, the width (W2) between the two ends of the large external arc (21) ranging from 0.05 to 10 μm, preferably from 0.06 to 8 μm, on average, and the height (H) of the large external arc (21) ranging from 0.015 to 8 μm, preferably from 0.03 to 6 μm, on average.

The dimensions mentioned above are obtained by calculating the mean of the dimensions of one hundred particles chosen on an image obtained with a scanning electron microscope.

Mention may be made, as concave particles in the form of portions of spheres which can be used according to the invention, of, for example:
particles composed of the crosslinked organosilicone TAK-110 (crosslinked methylsilanol/silicate polymer) from Takemoto Oil & Fat, with the shape of a bowl, with a width of 2.5 μm, a height of 1.2 μm and a thickness of 150 nm (particles sold under the name NLK-506 by Takemoto Oil & Fat);
particles composed of the crosslinked organosilicone TAK-110 (crosslinked methylsilanol/silicate polymer) from Takemoto Oil & Fat, with the shape of a bowl, with a width of 0.8 μm, a height of 0.4 μm and a thickness of 130 nm (particles sold under the name NLK-515 by Takemoto Oil & Fat);
particles composed of the crosslinked organosilicone TAK-110 (crosslinked methylsilanol/silicate polymer) from Takemoto Oil & Fat, with the shape of a bowl, with a width of 7 μm, a height of 3.5 μm and a thickness of 200 nm (particles sold under the name NLK-510 by Takemoto Oil & Fat).

These particles have the INCI name: Methylsilanol/silicate crosspolymer.

Advantageously, the concave silicone particles have a mean diameter of less than or equal to 5 μm, in particular ranging from 0.1 μm to 5 μm, preferably ranging from 0.2 to 5 μm, more preferably ranging from 0.5 to 4 μm and preferably again ranging from 0.5 to 3 μm.

These particles make possible, in addition to the reduction, indeed even the elimination, of the sticky feel, the optimization of the properties of slip, of spreading and of comfort of the composition according to the invention.

The silicone particles of annular shape are preferably chosen from those described and synthesized in Patent Application US-A-2006/0089478. They exhibit a mean external diameter of 0.05 to 15 μm and a mean internal diameter of 0.01 to 10 μm, the difference between the mean external diameter and the mean internal diameter being from 0.04 to 5 μm.

They exhibit a polysiloxane network comprising siloxane units of formulae (1), (2), (3), (4), (5) and (6):

| | |
|---|---|
| SiO_{4/2} | (1) |
| Si(OH)^{3/2} | (2) |
| R¹SiO_{3/2} | (3) |
| R²SiO_{3/2} | (4) |
| R³(SiOH)_{2/2} | (5) |
| R⁴(SiOH)_{2/2} | (6) |

in which:
R¹ and R³ denote unreactive hydrocarbon groups, in particular alkyl, cycloalkyl, aryl, alkylaryl or aralkyl groups, preferably C₁-C₃ alkyl groups, in particular methyl, ethyl or propyl groups and preferably a methyl group,
R² and R⁴ each denote a hydrocarbon group chosen from the acryloyloxy, methacryloyloxy, vinyl or mercapto groups;
   the siloxane units of formula (1)/siloxane units of formulae (2), (3), (4), (5) and (6) molar ratio being from 20/80 to 50/50;
   the siloxane units of formulae (2), (3) and (4)/siloxane units of formulae (5) and (6) molar ratio being from 50/50 to 75/25;
   the siloxane units of formulae (3) and (5)/siloxane units of formulae (4) and (6) molar ratio being from 20/80 to 60/40.

Mention may be made, as acryloyloxy group, of a 2-methacryloyloxyethyl group or a 3-acryloyloxypropyl group.

Mention may be made, as (meth)acryloyloxy group, of a 3-methacryloyloxypropyl group or a 3-acryloyloxypropyl group.

Mention may be made, as mercaptoalkyl group, of a mercaptopropyl or mercaptoethyl group.

Mention may be made, as vinyl group, of the allyl, isopropenyl or 2-methylallyl groups.

The concave or annular silicone particles can be present in the composition according to the invention in a content ranging from 0.1 to 20% by weight and preferably ranging from 0.5 to 10% by weight, with respect to the total weight of the composition.

Foaming Surfactants

The cleaning composition according to the invention comprises one or more foaming surfactants which will contribute to the cleaning nature of the composition. This or these foaming surfactants can be chosen from non-ionic surfactants, anionic surfactants, cationic surfactants, amphoteric and zwitterionic surfactants and the mixtures of these.

The foaming surfactants are detergents and differ from emulsifying surfactants in the value of their HLB (Hydrophilic Lipophilic Balance), the HLB being the ratio between the hydrophilic part and the lipophilic part of the molecule. The term “HLB” is well known to a person skilled in the art and is described, for example, in “The HLB system. A time-saving guide to Emulsifier Selection” (published by ICI Americas Inc; 1984).

For emulsifying surfactants, the HLB generally ranges from 3 to 8, for the preparation of water-in-oil (W/O) emulsions, and from 8 to 18, for the preparation of oil-in-water (O/W) emulsions, whereas foaming surfactants generally have an HLB of greater than 18 and better still of greater than 20.

The foaming surfactant or surfactants have to be present in an amount sufficient for the composition to have a foaming effect. The amount (as active material) of foaming surfactant(s) in the composition according to the invention can range, for example, from 1 to 50% by weight, preferably from 2 to 40% by weight, in particular from 2 to 35% by weight, more especially from 5 to 30% by weight, and even from 5 to 25% by weight, with respect to the total weight of the composition.

a) The nonionic surfactants can be chosen, for example, from alkylpolyglucosides (APGs), maltose esters, polyglycerolated fatty alcohols, glucamine derivatives, such as 2-ethylhexyloxycarbonyl-N-methylglucamine, and their mixtures.

Use is preferably made, as alkylpolyglucosides, of those comprising an alkyl group comprising from 6 to 30 carbon atoms and preferably from 8 to 16 carbon atoms and comprising a hydrophilic (glucoside) group preferably comprising 1, 2 or 3 glucoside units. Mention may be made, as alkylpolyglucosides, for example, of decyl glucoside (Alkyl C9/C11 polyglucoside (1.4)), such as the product sold under the name Mydol 10® by Kao Chemicals, the product sold under the name Plantaren 2000 UP® by Cognis and the product sold under the name Oramix NS 10® by Seppic; caprylyl/capryl glucoside, such as the product sold under the name Oramix CG 110® by Seppic; lauryl glucoside, such as the products sold under the names Plantaren 1200 N® and Plantacare 1200® by Cognis; coco glucoside, such as the product sold under the name Plantacare 818/UP® by Cognis; and their mixtures.

The maltose derivatives are, for example, those described in the document EP-A-566 438, such as 6′-(O-octanoyl)-D-maltose, or alternatively 6′-(O-dodecanoyl)-D-maltose, described in the document FR-2 739 556.

Mention may be made, among polyglycerolated fatty alcohols, of polyglycerolated dodecanediol (3.5 mol of glycerol), a product sold under the name Chimexane NF® by Chimex.

b) The anionic surfactants can be chosen, for example, from soaps (salts of fatty acids), carboxylates, acylamino acids, amidoether carboxylates, alkyl polyaminocarboxylates, alkyl ether sulphates, such as sodium laureth sulphates, alkylsulphonates, isethionates, alkyl methyltaurates, alkyl sulphosuccinates, alkyl sulphoacetates, alkyl phosphates (mono- or dialkyl phosphates), their salts and their mixtures.

Soaps are obtained from a fatty acid which is partially or completely saponified (neutralized) by a basic agent. These are soaps of alkali metals or alkaline earth metals or of organic bases. Use may be made, as fatty acids, of saturated, linear or branched, fatty acids comprising from 8 to 30 carbon atoms and preferably comprising from 8 to 22 carbon atoms. This fatty acid can in particular be chosen from palmitic acid, stearic acid, myristic acid, lauric acid and their mixtures.

Use may be made, as basic agents, for example, of alkali metal hydroxides (sodium hydroxide and potassium hydroxide), alkaline earth metal hydroxides (for example, magnesium hydroxide), ammonium hydroxide or also organic bases, such as triethanolamine, N-methylglucamine, lysine and arginine.

The soaps can in particular be fatty acid alkaline salts, the basic agent being an alkali metal hydroxide and preferably potassium hydroxide (KOH).

The amount of basic agent must be sufficient for the fatty acid to be at least partially neutralized.

Mention may in particular be made, as carboxylates, of alkyl glycol carboxylic acids (or 2-(2-hydroxyalkyloxy)acetic acids) and their salts, such as, for example, sodium lauryl glycol carboxylate, sold under the names Beaulight SHAA® or Beaulight LCA-25N® by Sanyo (CTFA name: Sodium Lauryl Glycol Carboxylate), or its corresponding acid form, sold under the name Beaulight SHAA (Acid Form)® by Sanyo.

Mention may be made, as acylamino acids, for example, of sodium cocoglycinate, sold by Ajinomoto under the name Amilite GCS 12, sodium lauryol glutamate, sold by Ajinomoto under the name Amisoft LS11, and sodium lauroylsarcosinate, sold by Seppic under the name Oramix L 30.

Mention may be made, as alkyl phosphates, for example, of lauryl phosphate, sold by Kao under the name MAP 20.

c) The amphoteric and zwitterionic foaming surfactants can be chosen, for example, from betaine derivatives, including amidopropyl betaines, amphoacetates and amphodiacetates, hydroxysultaines and their mixtures.

Mention may be made, as betaine derivatives, for example, of coco betaine, such as the product sold under the name Dehyton AB-30® by Cognis; lauryl betaine, such as the product sold under the name Genagen KB® by Clariant; oxyethylenated lauryl betaine (10 OE), such as the product sold under the name Lauryl Ether (10 OE) Betaine® by Shin Nihon Rica; oxyethylenated stearyl betaine (10 OE), such as the product sold under the name Stearyl Ether (10 OE) Betaine® by Shin Nihon Rica; cocamidopropyl betaine, for example sold under the name Velvetex BK 35® by Cognis; undecylenamidopropyl betaine, for example sold under the name Amphoram U® by Ceca; and their mixtures.

Mention may be made, as amphoacetates and amphodiacetates, for example, of N-disodium N-cocoyl-N-carboxymethoxyethyl-N-(carboxymethyl)ethylenediamine (CTFA name: disodium cocoamphodiacetate), such as the product sold under the name Miranol C2M Concentrate NP® by Rhodia Chimie; N-sodium N-cocoyl-N-hydroxyethyl-N-(carboxymethyl)ethylenediamine (CTFA name: sodium cocoamphoacetate), and their mixtures.

According to one embodiment of the invention, a surfactant suitable for the invention can be chosen from alkylpolyglucosides, betaine derivatives, alkyl glycol carboxylic acids and their salts, soaps, alkyl ether sulphates, alkyl phosphates, amphodiacetates, amphoacetates, alkylglycinates, acylglutamates, acylsarcosinates and their mixtures.

According to a specific embodiment of the invention, the foaming surfactant can be chosen in particular from decyl glucoside, cocoyl glucoside, sodium lauryl ether sulphate, cocoyl betaine, lauryl betaine, cocoamidopropyl betaine, lauramidopropyl betaine, lauryl glycol carboxylate, cocoampho(di)acetate, lauroampho(di)acetate, potassium lauryl phosphate, lauric acid salts, myristic acid salts, stearic acid salts, palmitic acid salts, and their mixtures.

Adjuvants

The compositions of the invention can also comprise adjuvants and active principles, including those conventionally used in the cosmetic or dermatological field. Mention may be made, for example, of water-soluble or fat-soluble adjuvants, such as thickening, dispersing or conditioning, anionic, nonionic, cationic or amphoteric and hydrophilic or lipophilic polymers; preservatives, sequestering agents (EDTA); antioxidants; fragrances; colouring materials, such as soluble dyes or encapsulated or nonencapsulated pigments; pearlescent agents; fillers with mattifying, tightening, whitening or exfoliating effects; sunscreens; hydrophilic or lipophilic cosmetic or dermatological active principles, such as water-soluble or fat-soluble vitamins, antiseptics, antiseborrhoeics, antimicrobials, such as benzoyl peroxide, salicylic acid, triclosan, azelaic acid or niacin (vit. PP); slimming agents, such as caffeine, and also optical brighteners; electrolytes; agents having the effect of improving the cosmetic properties of the skin or also porous or nonporous and spherical or nonspherical solid particles of any size.

The amounts of these various adjuvants are those conventionally used in the field under consideration, for example from 0.01 to 20% of the total weight of the composition.

These adjuvants, depending on their nature, can be introduced into the fatty phase or into the aqueous phase.

Of course, a person skilled in the art will take care to choose the optional additive or additives to be added to the composition according to the invention and their amounts so that the advantageous properties intrinsically attached to the composition in accordance with the invention are not, or not substantially, detrimentally affected by the envisaged addition.

The composition can comprise, as exfoliating fillers, exfoliating or scrubbing particles of mineral, vegetable or organic origin. Thus, use may be made, for example, of polyethylene beads or powder, nylon powder, poly(vinyl chloride) powder, pumice, ground materials derived from apricot kernels or walnut shells, sawdust, glass beads, alumina, and their mixtures. These particles can be present in an amount ranging from, for example, 0.5 to 30% by weight, preferably from 1 to 20% by weight and better still from 1 to 10% by weight, with respect to the total weight of the composition. When the composition comprises exfoliating particles, it can constitute in particular a composition for scrubbing the skin of the face or of the body.

The composition according to the invention can constitute in particular a cosmetic composition for cleaning keratinous substances, in particular skin and hair, and in particular a foaming cosmetic composition which can in particular be rinsed out after application to the skin or hair.

Another subject-matter of the invention is the cosmetic use of the composition as defined above for cleaning keratinous substances, in particular the skin and hair and very particularly the skin.

Another subject-matter of the invention is a method for cleaning the skin which consists in applying, to the skin, a composition as defined above and in rinsing the skin.

The examples which follow serve to illustrate the invention without, however, exhibiting a limiting nature. The amounts are expressed as percentage by weight of starting material. The compounds of the compositions according to the invention are, as the case may be, cited with chemical names or with INCI (International Cosmetic Ingredient) names.

### Example 1 According to the Invention and Comparative Example 1

| | Example 1 | |
|---|---|---|
| | according to | Comparative |
| | the invention | Example 1 |
| Lauryl phosphate (1) | 6.5 | 6.5 |
| Decyl glucoside (2) | 6.5 | 6.5 |
| Polyquaternium-7 (3) | 0.5 | 0.5 |
| PEG-60 hydrogenated | 0.2 | 0.2 |
| castor oil (4) | | |
| PEG-200 glyceryl stearate (5) | 0.5 | 0.5 |
| Potassium hydroxide (KOH) | 1.7 | 1.7 |
| Sodium chloride | 0.1 | 0.1 |
| EDTA | 0.05 | 0.05 |
| Sorbitol | 3.5 | 3.5 |
| Imidazolidinylurea | 0.2 | 0.2 |
| Methylparaben | 0.2 | 0.2 |
| Glycerol | 3.5 | 3.5 |
| Methylsilanol/silicate | 2.5 | — |
| crosspolymer (6) | | |
| Water | q.s. for | q.s. for |
| | 100% | 100% |

(1) MAP 20 ® from Kao Chemicals
(2) Mydol 10 ® from Kao Chemicals
(3) Merquat S ® from Calgon
(4) Cremophor RH 60 from BASF
(5) Simulsol 220 TM from Seppic
(6) NLK 506 from Takemoto Oil & Fat

Sensory Performance

The quality of the foam developed was evaluated according to the protocol described below.

Before any use of the product, the hands are washed with household soap and then suitably rinsed and dried. The protocol used is then as follows:

1—the hands are rendered wet by passing them under running water and are shaken three times to roughly dry them,

2—1 g of product is placed in the hollow of one of the hands,

3—the product is worked between both palms for 10 seconds,

4—2 ml of water are added and the product is again worked for 10 seconds,

5—2 ml of water are added and the product is again worked for 10 seconds,

6—the quality of the foam is evaluated according to the criteria defined,

7—the hands are rinsed under water,

8—they are wiped dry.

The criteria of foam quality (stage 6) are graded on a scale from 0 to 10:
The foam volume: the grade assigned increases as the volume increases.
The size of the bubbles composing the foam: the grade assigned increases as the bubbles become larger.
The staying power of the foam: the grade assigned increases as the foam becomes more elastic.
The softness of the foam: the grade assigned increases as the foam becomes more slippery and softer.

The evaluation panel was composed of 3 trained experts and the mean of the three grades was taken, which made it possible to compare the compositions according to the criteria.

| | Example | |
|---|---|---|
| | according to | Comparative |
| Foam quality | the invention | Example |
| Volume | 7 | 6.3 |
| Size of the bubbles | 3.7 | 4.2 |
| Staying power | 7.5 | 6.5 |
| Softness of the foam | 9.3 | 8.7 |

The above table shows that the example according to the invention, which comprises hollow organosilicone spheres, exhibits a superior foam quality, in particular a denser, softer and slightly finer (smaller size of the bubbles) and more voluminous foam.

### Example 2 According to the Invention

### Cream Soap

| | Example 2 |
|---|---|
| Lauric acid | 3 |
| Myristic acid | 20 |
| Stearic acid | 6 |
| Glyceryl stearate | 2 |
| Coco glucoside (7) | 1 |
| Potassium hydroxide (KOH) | 6.2 |
| Glycerol | 14 |
| PEG-8 | 7 |
| Methylsilanol/silicate | 3 |
| crosspolymer (6) | |
| Preservatives | 0.9 |
| Water | q.s. for 100% |

(6) NLK 506 from Takemoto Oil & Fat
(7) Plantacare 818 UP from Cognis

### Examples 3 and 4 According to the Invention

### Foaming Gel for the Face

| | Example 3 | Example 4 |
|---|---|---|
| Coco betaine (8) | 6.5 | — |
| Sodium cocoylglycinate (9) | 6.5 | — |
| Coco glucoside (7) | — | 6 |
| Sodium lauryl ether | — | 4 |
| sulphate (10) | | |
| PEG-150 pentaerythrityl | 1 | 1.5 |
| tetrastearate (11) | | |
| PEG-120 methylglucose | 2 | 1 |
| dioleate (12) | | |
| PEG-14M (13) | — | 1 |
| Methylsilanol/silicate | 10 | 5 |
| crosspolymer (6) | | |
| Water | q.s. for | q.s. for |
| | 100% | 100% |

(6) NLK 506 from Takemoto Oil & Fat
(7) Plantacare 818 UP from Cognis
(8) Dehyton AB 30 from Cognis
(9) Amilite GCS 12 from Ajinomoto
(10) Texapon AOS 225 UP from Cognis
(11) Crothix from Croda
(12) Glucamate DOE 120 from Noveon
(13) Polyox WSR205 from Amerchol

### Example 5 According to the Invention

### Shampoo

| | Example 5 |
|---|---|
| Sodium lauryl ether sulphate (10) | 15.5 |
| Cocoamphodiacetate (14) | 3.2 |
| Polquaternium-6 (15) | 0.6 |
| Sodium cetearyl sulphate | 0.75 |
| Oxyethylenated decyl alcohol (C₁₀/C₁₂/C₁₄) | 0.5 |
| Methylsilanol/silicate crosspolymer (6) | 5 |
| Citric acid | q.s. pH 5 |
| Demineralized water, q.s. | q.s. 100 |

(6) NLK 506 from Takemoto Oil & Fat
(10) Texapon AOS 225 UP from Cognis
(14) Miranol C2M Conc. from Rhodia
(15) Merquat ® 100 from Calgon

### Example 6 According to the Invention and Comparative Examples 2 and 3

| | Example | | |
|---|---|---|---|
| | according | | |
| | to the | Comparative | Comparative |
| Composition | invention | Example 2 | Example 3 |
| Lauryl phosphate (1) | 6.5 | 6.5 | 6.5 |
| Potassium hydroxide | 1.7 | 1.7 | 1.7 |
| (KOH) | | | |
| Disodium EDTA | 0.0495 | 0.0495 | 0.0495 |
| Decyl glucoside (2) | 6.5 | 6.5 | 6.5 |
| Polyquaternium-7 (3) | 0.5 | 0.5 | 0.5 |
| PEG-60 hydrogenated | 0.2 | 0.2 | 0.2 |
| castor oil (4) | | | |
| PEG-200 glyceryl | 0.5 | 0.5 | 0.5 |
| stearate (5) | | | |
| Sodium chloride | 0.1 | 0.1 | 0.1 |
| Sorbitol | 3.5 | 3.5 | 3.5 |
| Preservatives | 0.4 | 0.4 | 0.4 |
| Glycerol | 3.5 | 3.5 | 3.5 |
| Ethanol | 0.4 | 0.4 | 0.4 |
| Fragrance | 0.2 | 0.2 | 0.2 |
| Methylsilanol/silicate | 1.25 | — | — |
| crosspolymer (6) | | | |
| Silica (7) | — | 1.25 | — |
| Water | q.s. for | q.s. for | q.s. for |
| | 100% | 100% | 100% |
| Foam volume | 9.3 | 8.5 | 7.5 |
| Size of the bubbles | 4.5 | 5.5 | 4.8 |
| Softness | 8.8 | 7.5 | 7.5 |

(1) MAP 20 ® from Kao Chemicals
(2) Mydol 10 ® from Kao Chemicals
(3) Merquat S ® from Calgon
(4) Cremophor RH 60 from BASF
(5) Simulsol 220 TM from Seppic
(6) NLK 506 from Takemoto Oil & Fat
(7) Aerosil 200 from Degussa-Hüls

The above table shows that the example according to the invention, which comprises hollow organosilicone spheres, exhibits a better foam quality than that of a composition not comprising it (Comparative Example 3) and than that of a composition comprising silica (Comparative Example 2), in particular a denser, softer and finer (smaller size of the bubbles) and more voluminous foam.

The above written description of the invention provides a manner and process of making and using it such that any person skilled in this art is enabled to make and use the same, this enablement being provided in particular for the subject matter of the appended claims, which make up a part of the original description, and including an aqueous composition for topical application comprising (1) at least one foaming surfactant, the amount of foaming surfactant(s) being sufficient for the composition to have a foaming effect, (2) concave or annular particles of silicone material, in particular in the form of portions of hollow spheres, and (3) at least 20% by weight of water, with respect to the total weight of the composition.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. An aqueous composition comprising:
(1) an effective amount of at least one foaming surfactant;
(2) concave and/or annular particles of silicone material; and
(3) at least 20% by weight of water, with respect to the total weight of the composition.

2. The composition according to claim 1, comprising concave particles of silicone material having a mean diameter of less than or equal to 10 μm.

3. The composition according to claim 1, comprising concave particles of silicone material having a mean diameter ranging from 0.1 μm to 8 μm.

4. The composition according to claim 1, comprising concave particles of silicone material in the form of portions of hollow spheres having a transverse cross section with the shape of a horseshoe or arch.

5. The composition according to claim 1, wherein the silicone material is a crosslinked polysiloxane with a three-dimensional structure comprising units of formula (I): SiO₂ and of formula (II): R¹SiO_{1.5}
in which R¹ denotes an organic group having a carbon atom directly connected to the silicon atom.

6. The composition according to claim 5, wherein R¹ is a C₁-C₄ alkyl group or a phenyl group.

7. The composition according to claim 5, wherein R¹ is a methyl group.

8. The composition according to claim 5, wherein R¹ is chosen from epoxy, (meth)acryloyloxy, alkenyl, mercaptoalkyl, aminoalkyl, haloalkyl, glyceroxy, ureido and cyano groups.

9. The composition according to claim 5, wherein the silicone material comprises units (I) and (II) according to a unit (I)/unit (II) molar ratio ranging from 30/70 to 50/50.

10. The composition according to claim 1, comprising concave particles made of silicone material obtained according to a process comprising:
(a) the introduction into an aqueous medium, in the presence of at least one hydrolysis catalyst and optionally of at least one surfactant, of a compound (III) of formula SiX₄ and of a compound (IV) of formula RSiY₃, where X and Y denote, independently of one another, a C₁-C₄ alkoxy group, an alkoxyethoxy group including a C₁-C₄ alkoxy group, a C₂-C₄ acyloxy group, an N,N-dialkylamino group including C₁-C₄ alkyl groups, a hydroxyl group, a halogen atom or a hydrogen atom and R denotes an organic group comprising a carbon atom connected directly to the silicon atom; and
(b) the operation in which the mixture resulting from stage (a) is brought into contact with an aqueous solution comprising at least one polymerization catalyst and optionally at least one surfactant, at a temperature of between 30 and 85° C., for at least two hours.

11. The composition according to claim 10, wherein, in stage (a), the molar ratio of the compound (III) to the compound (IV) ranges from 30/70 to 50/50.

12. C The composition according to claim 10, wherein the ratio by weight of the water to the total of the compounds (III) and (IV) ranges from 10/90 to 70/30 in stage (a).

13. The composition according to claim 10, wherein R is a C₁-C₄ alkyl group or a phenyl group.

14. The composition according to claim 10, wherein R is a methyl group.

15. The composition according to claim 10, wherein R is chosen from epoxy, (meth)acryloyloxy, alkenyl, mercaptoalkyl, aminoalkyl, haloalkyl, glyceroxy, ureido and cyano groups.

16. The composition according to claim 1, comprising concave particles formed of a smaller internal arc, of a larger external arc and of segments which connect the ends of the respective arcs, the width between the two ends of the small internal arc ranging from 0.01 to 8 μm on average, the width between the two ends of the large external arc ranging from 0.05 to 10 μm on average, and the height of the large external arc ranging from 0.015 to 8 μm on average.

17. The composition according to claim 1, comprising particles of annular shape that exhibit a mean external diameter of 0.05 to 15 μm and a mean internal diameter of 0.01 to 10 μm, the difference between the mean external diameter and the mean internal diameter being from 0.04 to 5 μm.

18. The composition according to claim 17, wherein the particles of annular shape exhibit a polysiloxane network comprising siloxane units of formulae (1), (2), (3), (4), (5) and (6):

| | |
|---|---|
| SiO_{4/2} | (1) |
| Si(OH)_{3/2} | (2) |
| R₁SiO_{3/2} | (3) |
| R₂SiO_{3/2} | (4) |
| R₃SiO_{3/2} | (5) |
| R₄SiO_{3/2} | (6) |

in which R₁ and R₃ denote alkyl, cycloalkyl, aryl, alkylaryl or aralkyl groups and R₂ and R₄ each denote a hydrocarbon group chosen from the acryloyloxy, methacryloyloxy, vinyl or mercapto groups;
the siloxane units of formula (1)/siloxane units of formulae (2), (3), (4), (5) and (6) molar ratio being from 20/80 to 50/50;
the siloxane units of formulae (2), (3) and (4)/siloxane units of formulae (5) and (6) molar ratio being from 50/50 to 75/25;
the siloxane units of formulae (3) and (5)/siloxane units of formulae (4) and (6) molar ratio being from 20/80 to 60/40.

19. The composition according to claim 1, wherein the concave and/or annular particles are present in a content ranging from 0.1 to 15% by weight, with respect to the total weight of the composition.

20. The composition according to claim 1, wherein the foaming surfactant is chosen from nonionic surfactants, anionic surfactants, cationic surfactants, amphoteric and zwitterionic surfactants and their mixtures.

21. The composition according to claim 1, wherein the foaming surfactant is chosen from alkylpolyglucosides, betaine derivatives, soaps of alkali metals or alkaline earth metals or of organic bases, alkyl glycol carboxylic acids and their salts, alkyl ether sulphates, alkyl phosphates, amphodiacetates, amphoacetates, alkylglycinates, acylglutamates, acylsarcosinates and their mixtures.

22. The composition according to claim 1, wherein the amount of foaming surfactant(s) ranges from 1 to 50% by weight with respect to the total weight of the composition.

23. The composition according to claim 1, wherein it comprises particles in the form of portions of hollow spheres

24. A method for cleaning the skin which comprises applying, to the skin, a composition according to claim 1 followed by rinsing the skin.
